(19) United States
(12) Patent Application Publication
(10) Pub. No.: US 2015/0279515 A1
(43) Pub. Date: Oct. 1, 2015

(54) CABLE AND HARNESS USING THE CABLE

(71) Applicant: Hitachi Metals, Ltd., Tokyo (JP)

(72) Inventors: Detian HUANG, Hitachi (JP); Takanobu WATANABE, Hitachi (JP); Kimika KUDO, Kitaibaraki (JP)

(21) Appl. No.: 14/625,289

(22) Filed: Feb. 18, 2015

(30) Foreign Application Priority Data
Mar. 27, 2014 (JP) 2014-066159

(51) Int. Cl. H01B 7/18; H01B 7/00; A61L 31/14; H01B 3/30; A61L 31/04; A61B 8/00; H01B 3/44

(52) U.S. Cl. CPC H01B 7/188; A61B 8/4444; H01B 7/0045; H01B 3/443; H01B 3/307; A61L 31/041; A61L 31/14

(57) ABSTRACT

A cable includes sheath layer at an outermost layer, the sheath layer including a medical insulating resin, and a buffer layer closely contacting the sheath layer inside the sheath layer. The buffer layer includes a rubber elasticity resin.

The present application is based on Japanese patent application No.2014-066159 filed on Mar. 27, 2014, the entire contents of which are incorporated herein by reference.

### BACKGROUND OF THE INVENTION

1. Field of the Invention

The invention relates to a cable such as a ultrasonograph probe cable used in medical applications, and a harness using the cable.

2. Description of the Related Art

A ultrasonograph probe cable is known that is provided with multiple core wires (coaxial lines and insulated wires), a binding tape bundling the core wires and a braided shield and a sheath layer provided in this order on the outer periphery of the binding tape.

In medical-use cables such as the ultrasonograph probe cable, a sheath layer of a medical insulating resin is used as the outermost layer.

The medical insulating resin is also called a medical resin or a medical grade resin. It is biocompatible (or highly biologically compatible), non-toxic and does not cause allergic symptoms such as inflammation upon contact with a living organism.

PVC (polyvinyl chloride) is known as the medical insulating resin and generally used for cables.

The related art to the invention may include JP-A-2013-232356.

### SUMMARY OF THE INVENTION

The cables with the sheath layer of the medical insulating resin such as PVC may have the problem that a wrinkle occurs on a surface of the sheath layer especially by being bent at a small bend radius since the elasticity of the sheath layer is low. Thus, the appearance of the cables may deteriorate.

They may also have the problem that a crack is derived from the wrinkle on the sheath layer by repeatedly bending a portion nearby the wrinkle.

Furthermore, they may have the problem that since the ultrasonograph probe cable is as long as 2.2 to 3 m, it is broken by being stepped on when a part thereof is fallen on the floor or it is caught by a surrounding structure etc.

It is an object of the invention to provide a cable that is less likely to deteriorate in appearance and less likely to be broken, as well as a harness using the cable.

(1) According to one embodiment of the invention, a cable comprises:
a sheath layer at an outermost layer, the sheath layer comprising a medical insulating resin; and
a buffer layer closely contacting the sheath layer inside the sheath layer,
wherein the buffer layer comprises a rubber elasticity resin.

In the above embodiment (1) of the invention, the following modifications and changes can be made.
(i) The medical insulating resin has a higher softening temperature than the rubber elasticity resin.
(ii) The sheath layer comprises a polyvinyl chloride, wherein the buffer layer comprises a urethane rubber.
(iii) A part of the cable comprises a coiled shape in a longitudinal direction of the cable by plastically deforming the rubber elasticity resin by heat treatment.
(iv) The cable further comprises a plurality of core wires, wherein the buffer layer and the sheath layer are sequentially formed so as to collectively cover the plurality of core wires, and wherein the plurality of core wires each comprise an insulating resin comprising a fluoropolymer.
(v) The cable further comprises a braided shield inside the buffer layer so as to collectively cover the plurality of core wires, wherein the braided shield comprises a wire comprising a tinsel copper.

(2) According to another embodiment of the invention, a harness comprises:
the cable according to the above embodiment (1); and
a terminal component on at least one end of the cable.

### EFFECTS OF THE INVENTION

According to one embodiment of the invention, a cable can be provided that is less likely to deteriorate in appearance and less likely to be broken, as well as a harness using the cable.

### BRIEF DESCRIPTION OF THE DRAWINGS

Next, the present invention will be explained in more detail in conjunction with appended drawings, wherein:

FIGS. 1A to 1C are illustration diagrams showing a cable in an embodiment of the present invention, wherein FIG. 1A is a cross sectional view, FIG. 1B is a side view and FIG. 1C is a front view; and

FIG. 2 is an illustration diagram showing a harness using the cable of FIGS. 1A to 1C.

### DETAILED DESCRIPTION OF THE PREFERRED EMBODIMENT

An embodiment of the invention will be described below in conjunction with the appended drawings.

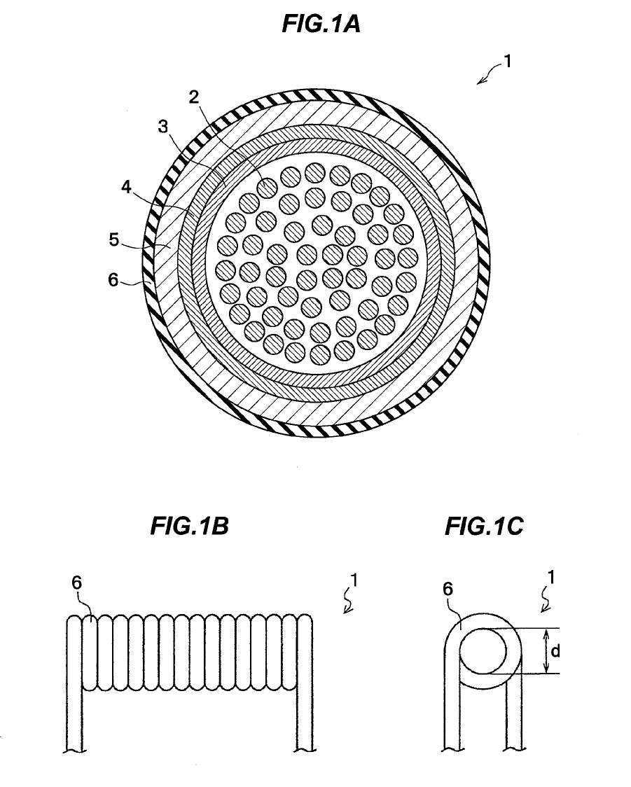

FIGS. 1A to 1C are illustration diagrams showing a cable in the present embodiment, wherein FIG. 1A is a cross sectional view, FIG. 1B is a side view and FIG. 1C is a front view.

As shown in FIG. 1A, in a cable 1, a sheath layer 6 formed of a medical insulating resin is provided as the outermost layer and a buffer layer 5 formed of a rubber elasticity resin is provided on the inner side of the sheath layer 6 so as to be tightly adhered to the sheath layer 6.

The cable 1 as a probe cable for ultrasonography will be described here.

The cable 1 is formed by covering plural (e.g., not less than one hundred) core wires 2 all together with a binding tape 3, a braided shield 4, the buffer layer 5 and the sheath layer 6 which are provided in this order on the plural core wires 2.

The core wires 2 comprise plural coaxial lines for signal transmission and plural insulated wires for power supply. The binding tape 3 is a resin tape for bundling plural core wires 2 and it is possible use, e.g., a PTFE (polytetrafluoroethylene) tape. The braided shield 4 is provided on the inner side of the buffer layer 5 such that the plural core wires 2 are covered all together.

The buffer layer 5, which is formed of a rubber elasticity resin, is provided on the outer periphery of the braided shield 4 and is tightly adhered to the sheath layer 6. When manufacturing the cable 1, the buffer layer 5 is provided and the sheath layer 6 is then formed by extrusion-molding of PVC on the outer periphery of the buffer layer 5, so the outer peripheral surface of the buffer layer 5 is softened by heat during the extrusion molding and adhesion between the buffer layer 5 and the sheath layer 6 is increased. Therefore, the sheath layer 6 is desirably formed of a medical insulating resin having a higher softening temperature than the rubber elasticity resin used to form the buffer layer 5.

In the present embodiment, a medical grade PVC is used as the medical insulating resin to form the sheath layer 6 and a urethane rubber is used as the rubber elasticity resin to form the buffer layer 5. The softening temperature of the PVC used to form the sheath layer 6 is preferably 5 to 10° higher than that of the urethane rubber used to form the buffer layer 5. The combination of the PVC and the urethane rubber is preferable to tightly adhere the buffer layer 5 to the sheath layer 6 since the PVC and the urethane rubber have the similar softening temperatures. The rubber elasticity resin used to form the buffer layer 5 is not limited to the urethane rubber and it is possible to use, e.g., silicon rubber, expanded PVC and polyphenylene oxide (allyl resin), etc.

Since the buffer layer 5 is provided so as to be tightly adhered to the sheath layer 6, the sheath layer 6 having a low elasticity follows and moves with the buffer layer 5 having a high elasticity and this allows generation of wrinkles on the sheath layer 6 to be suppressed. In addition, since the bend radius of the cable 1 is kept large by the buffer layer 5 when a bending force is applied to the cable 1, it is possible to suppress not only generation of wrinkles on the sheath layer 6 but also damages to the core wires 2 located inward.

Furthermore, even when the cable 1 is stepped on or caught by a surrounding structure, etc., the buffer layer 5 serves to protect the core wires 2 located inward and it is thus possible to prevent wire breakage in the cable 1.

Meanwhile, in the present embodiment, the rubber elasticity resin constituting the buffer layer 5 is plastically deformed by heat treatment, and the cable 1 is thereby shaped into a coiled shape (a curled shape or a spring shape) at least at a portion in the longitudinal direction.

In the present embodiment, the urethane rubber is used to form the buffer layer 5. Therefore, the cable 1 having the sheath layer 6 is wound around a mandrel and is then heat-treated at a temperature of 160 to 190° C. for 5 to 15 minutes so that the buffer layer 5 formed of the urethane rubber plastically deforms, thereby deforming the cable 1 into a coiled shape.

A fluoropolymer having high heat resistance is desirably used as an insulating resin (insulating sheath) of the core wire 2 so that deterioration due to heat treatment does not occur at the time of forming a coiled shape. When the buffer layer 5 is formed of the urethane rubber, PFA (tetrafluoroethylene perfluoroalkyl vinyl ether copolymer) or FEP (tetrafluoroethylene hexafluoropropylene copolymer), etc., rated at 200 degrees can be used as the insulating resin for the core wire 2.

Meanwhile, it is desirable to use tinsel copper as strands constituting the braided shield 4 so that wire breakage does not occur at the time of forming the coiled shape. The tinsel copper here is a strand formed by spirally winding a copper foil around a center thread formed of polyester or aramid, etc.

An inner diameter d of the coiled shape (i.e., an outer diameter of the mandrel used for forming the coiled shape) is not less than 3 times and not more than 5 times the outer diameter of the cable 1. This is because, when the inner diameter d is less than 3 times the outer diameter of the cable 1, the cable 1 is largely twisted at the time of being pulled and is likely to be broken. On the other hand, when it is more than 5 times, it is difficult to maintain the coiled shape. The outer diameter of the cable 1 is, e.g., 7 to 9 mm.

Since the cable is shaped into a coiled shape at least at a portion in the longitudinal direction, the coiled portion is stretched and thus reduces a load applied to the cable 1 even when tension is instantaneously applied to the cable 1 at the time of, e.g., operating a probe head, which allows breakage of the cable 1 to be prevented.

In addition, since the cable is shaped into a coiled shape at least at a portion in the longitudinal direction, it is possible to extend the cable 1 only when necessary and this allows storability of the cable 1 to be improved while providing enough cable length. In addition, in contrast to conventional cables which are, e.g., wound around an arm so as not to fall on the floor, it is not necessary to do so in the present embodiment since the cable 1 remains compressed when it is not necessary to be long and it is thus possible to significantly improve operability.

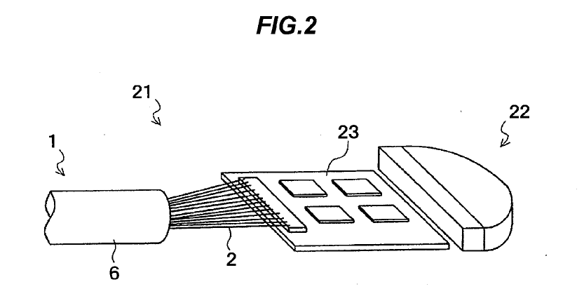

As shown in FIG. 2, a harness 21 in the present embodiment is composed of the cable 1 in the present embodiment and a probe head 22 as a terminal component provided on at least one of end portions of the cable 1. The core wires 2 of the cable 1 are connected to an internal circuit board 23 which comprises a PCB (printed circuit board) or a FPC (flexible printed circuit) and is located inside the probe head 22.

Although the terminal component of the cable 1 is described as a probe head in the present embodiment, it is not limited thereto. The terminal component may be configured to have, e.g., only the circuit board such as PBC or FPC or may be a connecter used for connection to another device, etc.

As described above, in the cable 1 of the present embodiment, the buffer layer 5 formed of a rubber elasticity resin is provided on the inner side of the sheath layer 6 formed of a medical insulating resin so as to be tightly adhered to the sheath layer 6.

Due to this configuration, the sheath layer 6 follows and moves with the buffer layer 5 having a high elasticity, and also, the bend radius of the cable 1 is kept large by the buffer layer 5 when a bending force is applied to the cable 1. As a result, it is possible to suppress generation of wrinkles on the sheath layer 6, thereby allowing deterioration in appearance of the cable 1 as well as cracks originated from the wrinkles on the sheath layer 6 to be suppressed.

In addition, providing the buffer layer 5 allows wire breakage to be prevented even when the cable 1 is stepped on or caught by a surrounding structure, etc., since the buffer layer 5 serves to protect the core wires 2 located inward.

The invention is not intended to be limited to the embodiment, and it is obvious that the various kinds of modification can be implemented without departing from the gist of the invention.

For example, although the cable shaped into a coiled shape at least at a portion in a longitudinal direction has been described in the present embodiment, the invention is applicable to straight type cables which do not have a coiled portion.

In addition, the cable 1 as a probe cable for ultrasonograph has been described in the embodiment, it is not limited thereto. The invention is applicable to various cables used in medical application.

What is claimed is:

1. A cable, comprising:
a sheath layer at an outermost layer, the sheath layer comprising a medical insulating resin; and
a buffer layer closely contacting the sheath layer inside the sheath layer,
wherein the buffer layer comprises a rubber elasticity resin.

2. The cable according to claim 1, wherein the medical insulating resin has a higher softening temperature than the rubber elasticity resin.

3. The cable according to claim 1, wherein the sheath layer comprises a polyvinyl chloride, and
wherein the buffer layer comprises a urethane rubber.

4. The cable according to claim 1, wherein a part of the cable comprises a coiled shape in a longitudinal direction of the cable by plastically deforming the rubber elasticity resin by heat treatment.

5. The cable according to claim 4, further comprising a plurality of core wires,
wherein the buffer layer and the sheath layer are sequentially formed so as to collectively cover the plurality of core wires, and
wherein the plurality of core wires each comprise an insulating resin comprising a fluoropolymer.

6. The cable according to claim 4, further comprising a braided shield inside the buffer layer so as to collectively cover the plurality of core wires,
wherein the braided shield comprises a wire comprising a tinsel copper.

7. A harness, comprising:
the cable according to claim 1; and
a terminal component on at least one end of the cable
